(19) United States
(12) Patent Application Publication
(10) Pub. No.: US 2026/0193579 A1
(43) Pub. Date: Jul. 9, 2026

(54) Bioreactor and Process for Microbial Gas Fermentation

(71) Applicant: PROTE-IN S.R.L., Bologna (IT)

(72) Inventor: Jian YU, Honolulu, HI (US)

(21) Appl. No.: 19/130,274

(22) PCT Filed: Jun. 30, 2022
(86) PCT No.: PCT/IT2022/000034

§ 371 Date: May 15, 2025

(51) Int. Cl. C12M 1/107; C12M 1/00; C12M 1/34; C12N 1/20

(52) U.S. Cl. CPC C12M 21/04; C12M 29/00; C12M 29/06; C12M 41/12; C12M 41/26; C12M 41/34; C12M 41/40; C12M 41/42; C12M 41/44; C12N 1/20

(57) ABSTRACT

A Y-type bioreactor for microbial gas fermentation includes an aeration chamber provided with elements for mixing a culture broth contained within. The bioreactor includes: at least a first and a second gas feeding chamber; at least a first duct configured for connecting an outlet terminal of the aeration chamber to at least one respective inlet of each of the first chamber and of the second chamber, and for introducing the culture broth coming from the aeration chamber into the first and second gas feeding chamber; at least a second duct; at least a third duct; at least one pump arranged in interception of the first duct for conveying the culture broth; and at least one outlet channel having its inlet arranged between a median height and the top of the aeration chamber, the outlet channel being configured for the continuous outflow of parts of the culture.

### CROSS-REFERENCE TO RELATED APPLICATION

This application is the National Stage of International Application No. PCT/IT2022/000034 filed Jun. 30, 2022, the disclosure of which is incorporated herein by this reference in its entirety.

### FIELD OF THE INVENTION

The present invention refers to a Y-type bioreactor for microbial gas fermentation, particularly for CO₂ fixation and conversion into valuable products such as dry cell mass, and single cell proteins (SCP).

### BACKGROUND OF THE INVENTION

Carbon dioxide (CO₂) is a major anthropogenic greenhouse gas (GHG) relevant to climate change. The United Nations Paris agreement set up a global goal of keeping temperature increases below 2° C. above the pre-industrial level. In response, the EU has set up a goal to reduce GHG emissions by 40% below 1990 levels by 2030. Governments around the world have also introduced regulations to promote carbon capture storage and utilization (CCSU) technologies by providing carbon tax credits. Carbon capture and utilization (CCU) is considered an important CO₂ mitigation strategy to support and compliment carbon capture and storage (CCS) for the abatement and sequestration of CO₂. CCU represents various pathways that utilize CO₂ as a raw material for production of value-added commodities. It provides economic incentives to industries, which can diversify their revenue portfolio by benefiting from additional or new commercial CO₂ utilization or by selling their captured CO₂ to other interested users. Biological CCU uses biological pathways to convert CO₂ to natural biomass and plays an important role within the energy, water, and food nexus. It could be an important resource management for water, energy, and food security.

Hydrogen-oxidizing bacteria such as Ralstonia eutropha are a type of chemoautotrophic microbial organisms that can convert CO₂ into desirable products by using hydrogen as the sole energy and reducing source and oxygen gas as the final electron acceptor [1]. Ralstonia eutropha is a representative chemoautotrophic bacterium that can grow on CO₂ as the carbon source. Both H₂ and O₂ gases can be conveniently generated from water electrolysis using renewable energy including solar, wind, hydro, and geothermal powers [2]. The microbial CO₂ fixation can therefore play a role in sustainable carbon capture and utilization.

Microbial growth on gas substrates (CO₂, H₂ and O₂) can produce valuable products from CO₂, water and renewable powers, which is beyond the capability of conventional green species such as green microalgae and plants that rely only on sunlight [2]. The bacterial dry mass contains ca. 70 wt % of proteins, particularly single cell proteins useful for aquaculture and animal feeds [3]. The essential amino acid composition of the microbial proteins is comparable to caseins (milk proteins) and superior to soybean proteins (a popular plant protein source). Food-grade proteins, peptides and amino acids can also be recovered by using proprietary downstream separation technologies.

The microbial gas fermentation can also be operated continuously regardless of the intermittency of sunlight, which significantly increases the process productivity. The carbon-to-products technology (C2P) can be applied in the areas that are not suitable for conventional agriculture. Specifically, growing crops needs a large area of arable land and consumes a large amount of fresh water due to evaporation loss. Conventional agriculture also loses up to 70% of nitrogen fertilizer to the environment because of running off. In contrast, the microbial gas fermentation consumes nitrogen nutrient and water with a very high efficiency as shown in the following two stoichiometric equations [1]. Fresh water is primarily converted into hydrogen (H₂) and oxygen (O₂) gases through water electrolysis and regenerated in formation of microbial biomass (CH_{1.68}O_{0.46}N_{0.24}). The water evaporation loss from the gas fermentation is quite low (<0.001 kg/kg dry cell mass), less than 1% water consumption of crop biomass production.

$$Water electrolysis : 7.77 {H}_{2} O = 7.77 {H}_{2} + 3.88 {O}_{2}$$

$$Microbial growth : {CO}_{2} + 7.77 {H}_{2} + 2.87 {O}_{2} + 0.24 {NH}_{3} = {CH}_{1.68} {O}_{0.46} {N}_{0.24} + 7.28 {H}_{2} O$$

The nitrogen nutrient (NH₃) is almost completely converted into organic nitrogen of cell mass. The single cell proteins account for ca. 70% of dry cell mass and could be a protein-rich source for aquaculture and animal feeds [3].

A major technical goal of industrial fermentation is the high productivity of the above described desired products. The productivity is often expressed as the amount of product produced per liquid volume per time, equal to a product concentration per time. It determines to a great extent the economic feasibility of the fermentation technology. For fast CO₂ fixation, the microbes should be fed with sufficient nutrients (nitrogen, phosphorous, minerals, etc.) as well as the gas substrates. Providing sufficient O₂ or H₂ is the most challenging task because of their very low solubility in aqueous solution.

Conventional gas fermentation uses gas mixtures and the gas is introduced into the bioreactor through a sparger at the bottom. The gas bubbles rise upwards in an aqueous medium solution, and depending on the bubbles' retention time only a partial amount of gas is dissolved in the solution and utilized by the microbes. As a result, the gas capture efficiency is low, generating a large amount of gas waste.

A typical gas mixture of 70% H₂, 20% O₂ and 10% CO₂ provides the microbial cells with relatively abundant H₂ and CO₂ but limited O₂ [6]. In the absence of dissolved oxygen (DO), the obligate aerobic microbes lose their metabolic activity and energy conversion efficiency.

Microbial gas fermentation in a conventional bioreactor therefore faces great challenges in operation safety, gas capture efficiency, and gas mass transfer for high fermentation productivity. First, a hydrogen and oxygen gas mixture has a high explosion risk unless the oxygen content is below 6% v/v or lower. Lack of the dissolved oxygen would lead to poor microbial growth and slow CO₂ fixation. Secondary, a low gas capture efficiency means a substantial amount of gas waste including CO₂ and H₂. Discharging CO₂ into the environment means a poor performance of CO₂ capture and utilization. H₂ is the most expensive gas substrate and its wastage not only increases the fermentation cost but also generates a potential explosive mixture of hydrogen and air. Finally, the gas mass transfer rate is restricted by the partial pressures of individual gases according to the Henry's law. Spreading gas bubbles in an aqueous solution provides a moderate volumetric transfer rate (k_{L}a) because of the moderate gas holdup.

Based on the above, it is very challenging to provide the microbes with the constant environment, and the balance of H₂, O₂ and CO₂ required for continuous growth at optimal conditions.

### BRIEF SUMMARY OF THE INVENTION

In view of the criticalities described above, the aim of the present invention is therefore to provide a bioreactor for microbial gas fermentation to maximize efficiency of conversion of CO₂ into desired products.

Another object of the invention is to provide a process for producing one or more of dry cell mass and single cell proteins from CO₂ with a higher yield and efficiency than the processes known so far.

Moreover, an object of the present invention is to provide a process which can be operated in a continuous mode.

Another object of the invention is to provide a process for the biological production of one or more of dry cell mass and single cell proteins by absorption and biological conversion of carbon dioxide, which is highly reliable and flexible in application, is relatively easy to provide and has competitive costs and almost no process waste.

This aim and these and other objects that will become better apparent hereinafter are achieved by a Y-type bioreactor for microbial gas fermentation comprising an aeration chamber provided with means for mixing a culture broth contained within, characterized in that said bioreactor comprises:
at least a first and a second gas feeding chamber suitable for containing, respectively, a first and a second gas,
at least a first duct configured for connecting an outlet terminal of said aeration chamber to at least one respective inlet of each of said first chamber and of said second chamber, and for introducing the culture broth coming from said aeration chamber into said first and second gas feeding chamber with consequent mixing of said culture broth with, respectively, said first and second gas,
at least a second duct configured for connecting an outlet terminal of said first gas feeding chamber to said aeration chamber,
at least a third duct configured for connecting an outlet terminal of said second gas feeding chamber to said aeration chamber,
at least one pump arranged in interception of said first duct, for the conveying of said culture broth,
at least one outlet channel, having its inlet arranged between a median height and the top of said aeration chamber, said outlet channel being configured for the continuous outflow of parts of said culture.

The aims and objects of the invention are also achieved by a process for producing one or more of dry cell mass and single cell proteins from CO₂ comprising the steps of:
(i) continuously providing an aqueous mineral solution to a culture of hydrogen-oxidizing microbes in the aeration chamber of a bioreactor according to the invention, obtaining a suspension,
(ii) providing H₂ gas to the first gas feeding chamber of said bioreactor,
(iii) providing CO₂ gas to the second gas feeding chamber of said bioreactor,
(iv) providing O₂ gas through the oxygen dispenser of said bioreactor,
(v) providing air or flue gas through the gas dispenser of said bioreactor,
(vi) continuously recirculating the suspension between the aeration chamber and said first and second gas feeding chamber wherein in the first gas feeding chamber droplets of culture are sprayed in a stagnant H₂ gas phase, and in the second gas feeding chamber, droplets of culture are sprayed in a CO₂ gas stagnant phase,
(vii) continuously discharging from the aeration chamber the suspension of microorganisms in aqueous mineral solution,
(viii) recovering dry cell mass or single cell proteins from the discharged suspension of step (vii).

### BRIEF DESCRIPTION OF THE DRAWINGS

Further characteristics and advantages of the invention will become more apparent from the description of a preferred but not exclusive embodiment of the bioreactor for bacterial fermentation according to the invention, illustrated by way of non-limiting example in the accompanying drawings, wherein:

FIG. 1 is a front schematic image in elevation of a first embodiment of bioreactor for bacterial fermentation according to the invention;

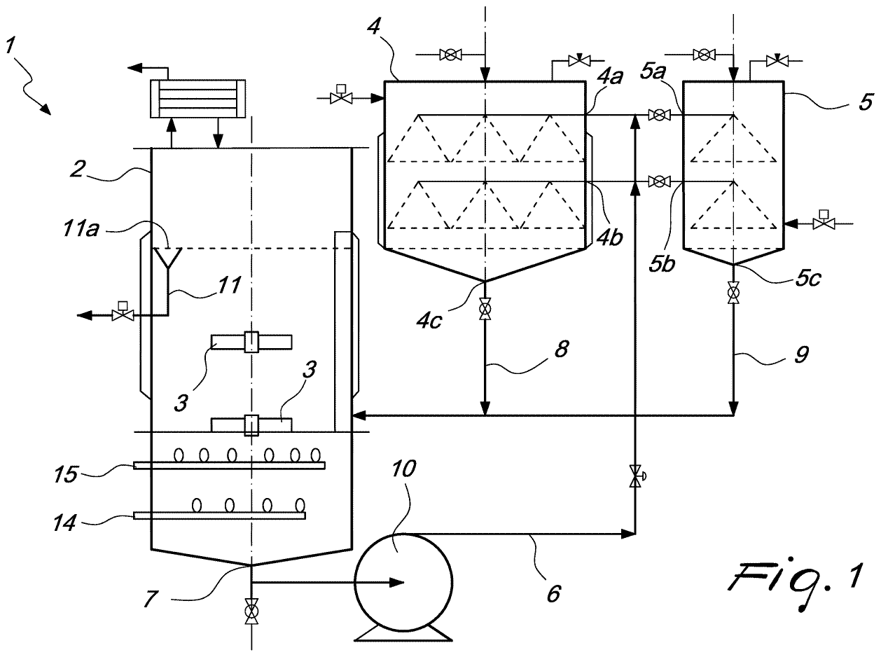

FIG. 2 is a plan view of the bioreactor of FIG. 1; and

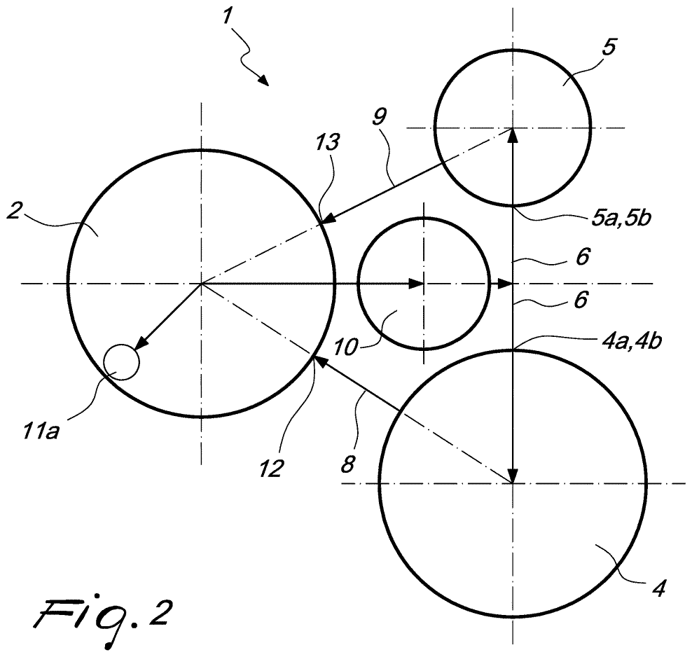

FIG. 3 is a front schematic image in elevation of another embodiment of bioreactor for bacterial fermentation according to the invention.

### DETAILED DESCRIPTION OF THE INVENTION

The process according to the invention seeks to contribute to the reduction of the concentration of carbon dioxide in the atmosphere, while producing valuable products.

The inventors have developed a novel Y-type bioreactor 1 that increases the gas transfer rates by using pure gases instead of the gas mixtures used in conventional gas fermentation.

In a first aspect the present invention refers to a Y-type bioreactor 1 for microbial gas fermentation comprising an aeration chamber 2 provided with means 3 for mixing a culture broth contained within. The bioreactor according to the invention comprises at least a first 4 and a second gas feeding chamber 5 suitable for containing, respectively, a first and a second gas, such as for example, H₂ and CO₂. The bioreactor further comprises:
at least a first duct 6 configured for connecting an outlet terminal 7 of the aeration chamber 2 to at least one respective inlet (4a, 4b, 5a, 5b) of each of the first chamber and of the second chamber 5, and for introducing the culture broth coming from the aeration chamber 2 into the first 4 and second gas feeding chamber 5 with consequent mixing of the culture broth with, respectively, the first and second gas,
at least a second duct 8 configured for connecting an outlet terminal 4c of the first gas feeding chamber 4 to the aeration chamber 2,
at least a third duct 9 configured for connecting an outlet terminal 5c of the second gas feeding chamber 5 to the aeration chamber 2,
at least one pump 10 arranged in interception of the first duct 6, for the conveying of the culture broth,
at least one outlet channel 11, having its inlet 11a arranged between a median height and the top of the aeration chamber 2, the outlet channel 11 being configured for the continuous outflow of parts of the culture.

In a preferred embodiment of the bioreactor according to the invention, the at least one first gas feeding chamber 4 is configured for receiving hydrogen (H₂) gas and for its mixing with the culture broth coming from the at least one first duct 6, and the second duct 8 is configured for the transfer of a mixture of hydrogen (H₂) and culture broth from the outlet terminal 4c of the first chamber 4 to at least one respective inlet 12 of the aeration chamber 2.

In another preferred embodiment of the bioreactor according to the invention, the at least one second gas feeding chamber 5 is configured for receiving carbon dioxide (CO₂) gas and for its mixing with the culture broth coming from the at least one first duct 6, and the third duct 9 is configured for the transfer of a mixture of dissolved carbon dioxide (CO₂) and culture broth from the outlet terminal 5c of the second chamber 5 to at least one respective inlet 13 of the aeration chamber 2.

Preferably, the aeration chamber 2 comprises an oxygen dispenser 14 and a gas dispenser 15 configured for the separate, independent, and uniform introduction of oxygen and a gas selected from air and flue gas into the culture broth present inside the aeration chamber 2. Preferably, the oxygen dispenser 14 is located at a lower altitude than the gas dispenser 15 selected for from air or and flue gas.

Preferably, the at least one first inlet 4a, 4b, of the first gas feeding chamber 4 is connected to at least one nozzle configured to nebulize the culture broth, comprising a microbial suspension and coming from the first duct 6, to the interior cavity delimited by the first gas feeding chamber 4.

Preferably, the at least one first inlet 5a, 5b, of the second gas feeding chamber 5 is connected to at least one nozzle configured to nebulize the culture broth, comprising a microbial suspension and coming from the first duct 6, to the interior cavity delimited by the second gas feeding chamber 5.

Preferably, the aeration chamber 2 comprises at least one device selected from probes, indicators and regulators referred to at least one parameter selected from fermentation temperature, pH, mixing speed of the culture broth, dissolved oxygen concentration, air flow rate, pressure inside the corresponding chamber, liquid level and the like.

The bioreactor according to the invention allows to conduct the fermentation without hydrogen and oxygen gas mixing in the bioreactor thus avoiding a potential explosive gas mixture. The hydrogen gas is dissolved in the aqueous medium solution in a closed chamber and the dissolved hydrogen molecules are delivered to an aeration chamber in which the obligate aerobic microbes utilize the dissolved hydrogen molecules to fixCO₂. The hydrogen gas capture efficiency is almost 100%. Furthermore, the hydrogen gas pressure and hence the hydrogen solubility is significantly increased in the closed chamber according to the Henry's law. The aqueous mineral solution or microbial slurry is spread into tiny liquid droplets though multiple nozzles, which generates a huge gas-liquid contact area for quick gas absorption. As a result, the hydrogen gas mass transfer rate is significantly increased for a high fermentation productivity. Moreover, the CO₂ gas is dissolved in the aqueous medium solution in a separate gas chamber and the dissolved CO₂ molecules are delivered to the microbes in the aeration chamber. The gas capture efficiency of CO₂ is almost 100%. Finally, the aeration chamber is equipped with two gas spargers, one for air and another for pure O₂. The oxygen mass transfer rate of pure O₂ is five times higher than that of air. It has been found that the dissolved oxygen concentration should be controlled within a range between a critical low level and an inhibitive high level. This dual oxygen supply design provides a great flexibility to control the dissolved oxygen concentration in the aqueous solution.

The aqueous mineral solution can be continuously added into and withdrawn from the bioreactor allowing the microbial organisms to grow on the dissolved gases under optimal conditions. The dry cell mass or single cell proteins are recovered from the slurry (suspension) discharged from the bioreactor.

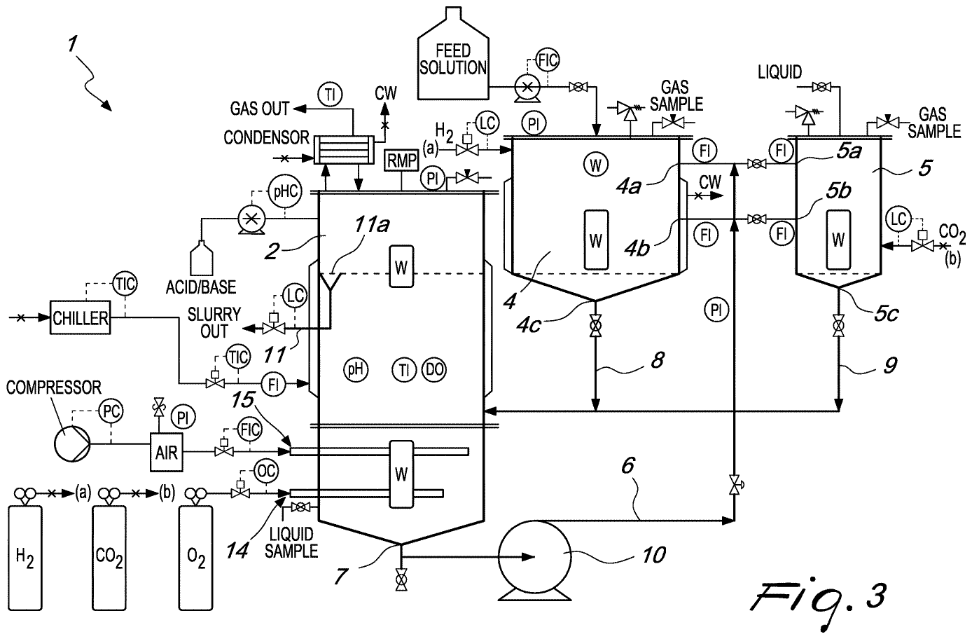

FIG. 3 represents an embodiment of the bioreactor according to the invention with auxiliary facilities: cooling water chiller, air compressor, and gas cylinders. Monitoring and controlling include: glass windows (W), pressure indication (PI), pressure control (PC), pressure and control indication (PIC), Flowrate indication (FI), flowrate control (FC), Flowrate indication and control (FIC), temperature indication (TI), temperature control (TC), temperature indication and control (TIC), pH indication (pH), pH indication and control (pHC), level control (LC), agitation speed indication and control (RMP), and dissolved oxygen concentration indication (DO) and control (OC).

In a second aspect, the present invention refers to a process for producing one or more of dry mass or single cell proteins from CO₂ comprising the steps of:
(i) continuously providing an aqueous mineral solution to a culture of hydrogen-oxidizing microbes in the aeration chamber 2 of a bioreactor according to the invention, obtaining a suspension,
(ii) providing H₂ gas to the first gas feeding chamber 4 of said bioreactor,
(iii) providing CO₂ gas to the second gas feeding chamber 5 of said bioreactor,
(iv) providing O₂ gas through the oxygen dispenser 14 of said bioreactor,
(v) providing air or flue gas through the gas dispenser 15 of said bioreactor,
(vi) continuously recirculating the suspension between the aeration chamber 2 and said first 4 and second gas feeding chamber 5 wherein in the first gas feeding chamber 4 droplets of culture are sprayed in a stagnant H₂ gas phase, and in the second gas feeding chamber 5, droplets of culture are sprayed in a CO₂ gas stagnant phase,
(vii) continuously discharging from the aeration chamber 2 the suspension of microorganisms in aqueous mineral solution,
(viii) recovering dry cell mass or single cell proteins from the discharged suspension of step (vii).

Preferably, throughout steps (i) to (vii) the culture in the aeration chamber 2 is maintained at a temperature of 30±1° C. and a pH of 7±0.5.

Preferably, the hydrogen-oxidizing microbe is a strain of Ralstona eutropha.

There are two types of CO₂ gas that could be used as the carbon source. In response to the increased concerns over climate change and a growing market of carbon credit, carbon capture and biofuel production such as ethanol fermentation generate quite pure CO₂ streams (>95% CO₂). This type of CO₂ gas should be converted to products with a very high efficiency (>90%) and little CO₂ being discharged into air. It is an engineering challenge because of the low solubility of CO₂ in aqueous solution. The second type of CO₂ gas is the flue gas discharged from various point sources such as power plants, steel makers, cement producers, and chemical processers. The carbon content of flue gas varies and is relatively low (<15% CO₂) because of the presence of a large amount of inert gas such as nitrogen. At the point sources, the flue gas is often discharged into air after washing and dedusting. A reasonable carbon capture efficiency is also desired for this type of gas waste as demanded by process decarbonization. The novel Y-type bioreactor can handle both types of CO₂ gas stream as the carbon feed. The bioreactor 1 according to the invention allows to obtain conversion of CO₂ with high productivity of desired products by circumventing issues related to the low solubility of O₂ and H₂ in aqueous solution and achieving high gas mass transfer rates by using pure gases, instead of the gas mixtures used in conventional gas fermentation. For instance, the oxygen mass transfer rate can be increased by 5 folds when a gas mixture (20% O₂) is replaced by pure oxygen gas (100% O₂).

The process according to the invention provides a very high productivity. When the cell density reaches a critical level determined by the gas mass transfer rates in bioreactor, the microbial cells can be harvested for single cell protein (SCP) production. With this fermentation control strategy, the aerobes do not suffer the limitation of dissolved oxygen even at a relatively high cell density.

### REFERENCES

[1] Jian Yu, Yue Lu (2019). Carbon dioxide fixation by a hydrogen-oxidizing bacterium: biomass yield, reversal respiratory quotient, stoichiometric equations and bioenergetics, Biochemical Engineering Journal.
[2] Jian Yu (2014). Bio-based products from solar energy and carbon dioxide, Trends in Biotechnology 32:5-10.
[3] Jian Yu (2018) Fixation of carbon dioxide by a hydrogen-oxidizing bacterium for value-added products. World Journal of Microbiology and Biotechnology.
[4] Yue Lu, Jian Yu (2017). Gas mass transfer with microbial CO₂ fixation and poly(3-hydrobybutyarte) synthesis in a packed bed bioreactor, Biochemical Engineering Journal, 122:13-21.
[5] Shimin Kang, Jian Yu (2015). Reaction routes in catalytic reforming of poly(3-hydroxybutyrate) into renewable hydrocarbon oil, RSC Adv., 2015, 5, 30005-30013.
[6] Yue Lu, Jian Yu (2017). Comparison analysis on the energy efficiencies and biomass yields in microbialCO2 fixation, Process Biochemistry, 62:151-160.

1-11. (canceled)

12. A Y-type bioreactor for microbial gas fermentation comprising an aeration chamber provided with means for mixing a culture broth contained within, the bioreactor comprising:
at least a first and a second gas feeding chamber suitable for containing, respectively, a first and a second gas;
at least a first duct configured for connecting an outlet terminal of said aeration chamber to at least one respective inlet of each of said first chamber and of said second chamber, and for introducing the culture broth coming from said aeration chamber into said first and second gas feeding chamber with consequent mixing of said culture broth with, respectively, said first and second gas;
at least a second duct configured for connecting an outlet terminal of said first gas feeding chamber to said aeration chamber;
at least a third duct configured for connecting an outlet terminal of said second gas feeding chamber to said aeration chamber;
at least one pump arranged in interception of said first duct, for the conveying of said culture broth; and
at least one outlet channel, having an inlet thereof arranged between a median height and a top of said aeration chamber, said outlet channel being configured for a continuous outflow of parts of said culture.

13. The bioreactor according to claim 12, wherein said at least one first gas feeding chamber is configured for receiving hydrogen gas and for mixing the hydrogen gas with said culture broth coming from said at least one first duct, said second duct being configured for a transfer of a mixture of hydrogen and culture broth from said outlet terminal of said first chamber to at least one respective inlet of said aeration chamber.

14. The bioreactor according to claim 12, wherein said at least one second gas feeding chamber is configured for receiving carbon dioxide gas and for mixing thereof with said culture broth coming from said at least one first duct, said third duct being configured for the transfer of a mixture of dissolved carbon dioxide and culture broth from said outlet terminal of said second chamber to at least one respective inlet of said aeration chamber.

15. The bioreactor according to claim 12, wherein said aeration chamber comprises an oxygen dispenser and a gas dispenser configured for a separate, independent, and uniform introduction of oxygen and a gas selected from air and flue gas into the culture broth present inside said aeration chamber.

16. The bioreactor according to claim 15, wherein said oxygen dispenser is located at a lower altitude than said gas dispenser selected for from air or and flue gas.

17. The bioreactor according to claim 12, wherein said at least one first inlet of said first gas feeding chamber is connected to at least one nozzle configured to nebulize said culture broth, comprising a microbial suspension and coming from said first duct, to an interior cavity delimited by said first gas feeding chamber.

18. The bioreactor according to claim 12, wherein said at least one first inlet of said second gas feeding chamber is connected to at least one nozzle configured to nebulize said culture broth, comprising a microbial suspension and coming from said first duct, to an interior cavity delimited by said second gas feeding chamber.

19. The bioreactor according to claim 12, wherein said aeration chamber comprises at least one device selected from a probe, indicator, or regulator, wherein the device refers to at least one parameter selected from the group consisting of: fermentation temperature; pH; mixing speed of the culture broth; dissolved oxygen concentration; air flow rate; pressure inside the corresponding chamber; and liquid level.

20. A process for producing single cell proteins from CO₂ comprising the steps of:
(i) continuously providing an aqueous mineral solution to a culture of hydrogen-oxidizing microbes in the aeration chamber of a bioreactor according to claim 12 and obtaining a suspension;
(ii) providing H₂ gas to the first gas feeding chamber of said bioreactor;
(iii) providing CO₂ gas to the second gas feeding chamber of said bioreactor;
(iv) providing O₂ gas through the oxygen dispenser of said bioreactor;
(v) providing air or flue gas through the gas dispenser of said bioreactor;
(vi) continuously recirculating the suspension between the aeration chamber and said first and second gas feeding chamber, wherein in the first gas feeding chamber droplets of culture are sprayed in a stagnant H₂ gas phase, and in the second gas feeding chamber droplets of culture are sprayed in a CO₂ gas stagnant phase;
(vii) continuously discharging from the aeration chamber the suspension of microorganisms in aqueous mineral solution; and
(viii) recovering dry cell mass or single cell proteins from the discharged suspension of step (vii).

21. The process of claim 20, wherein throughout steps (i) to (vii) the culture in the aeration chamber is maintained at a temperature of 30±1° C. and a pH of 7±0.5.

22. The process of claim 20, wherein the hydrogen-oxidizing microbe is a strain of Ralstona eutropha.
